(19) United States
(12) Patent Application Publication
(10) Pub. No.: US 2020/0237705 A1
(43) Pub. Date: Jul. 30, 2020

(54) METHOD TO TREAT ANTIMICROBIAL RESISTANT CANDIDA

(71) Applicant: Hennepin Life Sciences, LLC, Plymouth, MN (US)

(72) Inventors: Patrick M. Schlievert, Iowa City, IA (US); Marnie L. Peterson, Jackson, WY (US)

(21) Appl. No.: 16/756,311

(22) PCT Filed: Oct. 11, 2018
(86) PCT No.: PCT/US18/55346

§ 371 Date: Apr. 15, 2020

Related U.S. Application Data
(60) Provisional application No. 62/572,669, filed on Oct. 16, 2017.

(51) Int. Cl. A61K 31/23; A61P 31/10; A61K 9/06; A61K 47/38; A61K 47/18; A61K 47/34

(52) U.S. Cl. CPC A61K 31/23; A61P 31/10; A61K 47/34; A61K 47/38; A61K 47/183; A61K 9/06

(57) ABSTRACT

This disclosure describes the ability of glycerol monolaurate, formulated both as a solution and as a nonaqueous gel, to kill antimicrobial resistant C. auris. Additionally, this disclosure describes the ability of glycerol monolaurate formulated as a non-aqueous gel to kill clinically isolated Candida species.

### BACKGROUND

Candida species of yeasts, including C. albicans, C. glabrata, C. krusei, C. parapsilosis, and C. pseudotropicalis have long been recognized as causes of large numbers of “yeast” infections in humans. These include, for example, both common vaginal yeast infections in women and thrush in the very young and immune compromised. More recently, Candida species have become the 4th leading cause of bloodstream infections. Generally, the dominant pathogenic Candida has always been C. albicans.

More recently, a new species of Candida has been identified as an emerging infectious disease pathogen, namely C. auris [Sears D, Schwartz B S. 2017. Candida auris: an emerging multidrug-resistant pathogen. Int J Infect Dis, doi:10.1016, j.ijid. 2017.08.017]. This Candida species of yeast tends to develop broad antimicrobial resistance to standard antimicrobials, unlike the other Candida species, creating challenges to clinically control and treat diseases associated with resistant C. auris or other resistant Candida species.

### SUMMARY

In one embodiment, this disclosure describes a method of treating an infected patient with an infection associate with an antimicrobial resistant Candida strain comprising the step of administering a composition comprising a pharmaceutically effective amount of glycerol monolaurate or a derivative to the infected patient for a sufficient amount of time to kill the resistant Candida strain.

In another embodiment, this disclosure describes a method of treating a biofilm of an antimicrobial resistant Candida strain in an infected patient comprising the step of administering a pharmaceutically effective amount of glycerol monolaurate or a derivative to the biofilm in the infected patient for a sufficient amount of time to clinically remedy the resistant Candida strain biofihn.

One embodiment of the present disclosure is a gel-based formulation comprising a composition that kills, or inhibits the growth of, one or more Candida species that cause, for example vaginal yeast infections and thrush, where the composition comprises about 0.0001-0.05 M of an accelerant selected from the group consisting of lactic acid, ascorbic acid, citric acid, ethylenediaminetetraacetic acid (ETDA), and combinations thereof, and about 10-100 mg/mL of GML.

In an exemplary embodiment, the gel-based formulation includes GML in an amount of about 10-100 mg/mL, preferably about 30-70 mg/mL. The gel-based formulation may also include a glycol, a cellulose derivative, a plant-derived oil, and/or petroleum jelly. Still further, the gel-based formulation may include an additional active material selected from an antibacterial, anti-viral, anti-fungal, anti-protozoan, or a combination thereof.

In still other embodiments, an accelerant and GML are combined with a topical gel comprising the following components:

a) about 73.55 w/w % propylene glycol;

b) about 25 w/w % polyethylene glycol 400;

c) about 1.25 w/w % hydroxyethyl cellulose or hydroxypropyl cellulose; and

d) about 1-25 w/w % saline and/or water.

Alternatively, the accelerant and GML are combined with a topical solution comprising substantially pure or about 100% w/w % plant-derived oil, petroleum jelly or derivatives thereof. In some these embodiments the plant-derived oil is selected from the group consisting of palm oil, olive oil, corn oil, and combinations thereof.

In other embodiments the gel-based formulation has a pH of about 4-4.5

The gel-based formulations of the present invention may be administered either before, simultaneous with, or after the administration of one or more supplementary materials. Supplementary materials can include, for example, anti-fungal materials, modulators of immune function, or antibiotics. In addition, an implant or indwelling device may be coated with a gel-based formulation of the present disclosure. Such a coated implant or device may be used in a method of treating or preventing a Candida-based infection in a patient when the implant or device is placed in a patient.

Compositions containing GML, one or more pharmaceutical excipients, and one or more gel-based formulations may also be included in various types of gels, creams, or foams.

### DESCRIPTION OF THE DRAWINGS

FIG. 1 is a graphical representation of the ability of various solutions comprising GML to kill Candida auris.

FIG. 2 is a graphical representation of the ability of GML Gel to kill multiple Candida clinical strains.

FIG. 3 is a graphical representation of the inability of a placebo gel that did not contain GML to kill multiple Candida clinical strains.

FIG. 4 is a graphical representation of the ability of GML Gel, but not a placebo gel, to prevent and remove Candida auris biofilms

### DETAILED DESCRIPTION

Glycerol monolaurate (GML) alone is reported to have the ability to kill selected Candida species, including C. albicans, C. glabrata, C. krusei, C. parapsilosis, and C. pseudotropicalis, as tested in vitro [Strandberg K L, Peterson M L, Lin Y C, Pack M C, Chase D J, Schlievert P M. 2010. Glycerol monolaurate inhibits Candida and Gardnerella vaginalis in vitro and in vivo but not Lactobacillus. Antimicrob Agents Chemother 54:597-601]. Additionally, this reported study disclosed evidence in humans that GML can reduce the numbers of Candida vaginally in women of menstrual age. U.S. Pat. No. 9,603,824 and U.S. Published patent application US 20170172968 also report that GML is active against Candida in vitro and in vivo; this patent and published application are both incorporated by reference in this application.

GML alone and in combination with a non-aqueous gel has never been tested for antimicrobial activity against C. auris. Additionally, GML, formulated as a non-aqueous gel, as a single antimicrobial agent has never been tested for activity against all Candida species.

This disclosure reports an assessment of the ability of GML alone to kill C. auris. Additionally, this disclosure reports an assessment of the ability of a GML formulated as a nonaqueous gel to kill all pathogenic Candida species

Glycerol monolaurate (GML) and GML-related compositions, together with suitable accelerants, in gel-based formulations may be applied to biological surfaces (skin and/or mucous membranes) to kill pathogenic Candida species, as well as inhibit production of exotoxins by pathogenic microorganisms, prevent inflammation and stabilize human cells to interfere with toxic reactions or infections, and select for beneficial bacteria such as lactobacilli and bifidobacteria.

The terms “active material” mean an antibacterial material, anti-fungal material, anti-viral material, anti-protozoan material, or combination thereof. Antibacterials for use with this disclosure, for example, include aminoglycosides, carbacephems, cephalosporins, glycopeptides, lincosamides, lipopetides, macrolides, monobactams, nitrofurans, penicillins, polypetides, quinolones, sulfuramides, and tetracyclines. Anti-fungal materials include, without limitation, those of the azole class, polyene class, or echinocandins class, nucleoside analogues, allylamines, griseofulvin, tolnaftate, or selenium compounds. Anti-viral materials include, for example and without limitation, acyclovir, ganciclovir, valganciclovir, abacavir, enofovir, lamivudine, emtricitabine, zidovudine, tenofovir, efavirenz, raltegravir, enfuvirdide, maraviroc, ribavirin, amantadine, rimantadine, interferon, oseltamivir, and zanamivir.

The term “administering” means clinically effective ways to delivering an active material described in this disclosure including, for example, by oral delivery, by topical application, or by injection.

The term “antimicrobial” means effective in preventing, inhibiting, or arresting the growth or pathogenic effects of a microorganism. “Microorganism” is used herein to mean any bacteria, virus, or fungus. In one embodiment, the formulations of this disclosure are used to prevent, inhibit, or arrest the growth, for example, C. auris.

The term “anti-viral” refers to inhibition of viral infection or virus replication, a reduction in the likelihood that a patient exposed to a virus will contract the viral disease or a reduction in the severity of the viral disease.

The term “biofilm” means an aggregate of microorganisms, usually bacterial, adhered to one another and growing on a surface. The microbial cells in the biofilm typically produce an extracellular matrix known as an extracellular polymeric substance. Often, this matrix and the density of the aggregate itself significantly increase the antibiotic resistance of the bacteria in the biofilm. Biofilms can be involved in known yeast infections such as vaginal infections and thrush.

The terms “cellulose derivative” refer to any a cellulose-based compound and may include, for example, hydroxyethyl cellulose, hydroxypropyl cellulose, methylcellulose, ethylcellulose, hydroxypropyl methyl cellulose, or cellulose acetate.

The term “derivative”, in some embodiments, means an active compound selected from the group consisting of Formula 1, Formula 2, and a combination of Formulas 1 and 2,

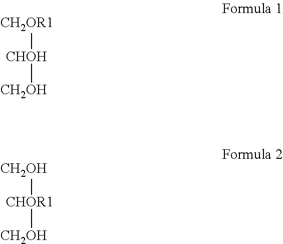

wherein R1 is: CO(CH₂)₁₀CH₃, and, in other embodiments, means an active compound selected from the group consisting of Formula 3 or Formula 4, and a combination of Formulas 3 and 4.

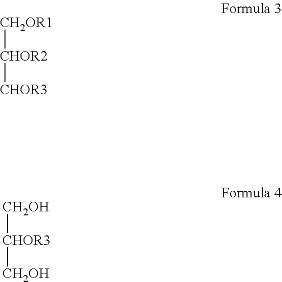

wherein R1 may be: hydrogen, CO(CH2)8CH3, CO(CH2)10CH3, or CO(CH2)12CH3;
R2may be: hydrogen, CO(CH2)8CH3, CO(CH2)12CH3, O(CH2)9CH3, O(CH2)11CH3, or O(CH2)13CH3, and

### R3 may be: CO(CH2)8CH3, CO(CH2)10CH3, CO(CH2)12CH3, O(CH2)9CH3, O(CH2)11CH3, or O(CH2)13CH3.

The terms “effective amount” refer to an amount that is sufficient to affect a beneficial or desired antimicrobial activity, including, without limitation, killing the microorganism or inhibiting microbial infection, growth or toxicity. An effective amount of GML is about up to 1 mg/mL, about up to 10 mg/mL, about up to 50 mg/mL, or about up to 100 mg/mL.

The terms “isolated compound” refer to a compound (e.g., GML or a related compound) that either has no naturally-occurring counterpart or has been separated or purified from components which naturally accompany it, e.g., in tissues such as pancreas, liver, spleen, ovary, testis, muscle, joint tissue, neural tissue, gastrointestinal tissue or tumor tissue, or body fluids such as blood, serum, or urine. Typically, a naturally occurring biological compound is considered “isolated” when it is at least 70%, by dry weight, free from other naturally-occurring organic molecules with which it is naturally associated. Preferably, a preparation of a compound for use in this disclosure is at least 80%, more preferably at least 90%, and most preferably at least 99%, by dry weight, that compound. The degree of isolation or purity can be measured by any appropriate method, e.g., column chromatography, polyacrylamide gel electrophoresis, or HPLC analysis. Since a compound (e.g., GML) that is chemically synthesized is, by its nature, separated from the components that naturally accompany it, the synthetic compound is by definition “isolated”. Isolated compounds, and supplementary materials useful for this disclosure, can be obtained, for example, by: (i) extraction from a natural source (e.g., from tissues or bodily fluids); (ii) where the compound or supplementary materials are proteins, by expression of recombinant nucleic acids encoding the proteins; or (iii) by standard chemical synthetic methods known to those in the art.

The term “kill” means a reduction in the number of detectable microorganisms of≥3 logs or more after contacting an active material described in this disclosure. If an initial inoculum or sample, for example, has approximately 10⁵ colony-forming units per milliliter (CFM/ml), it would be considered a kill if it is contacted for about 15 minutes with an active material as described in this disclosure and the amount of detectable microorganisms in the inoculum or sample is determined to be less than 10 CFU/ml.

The terms “pharmaceutically acceptable excipient” mean an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The terms “pharmaceutically acceptable topical carrier” refer to a material, diluent, or vehicle that can be applied to skin or mucosal surfaces without undue toxicity, irritation, or allergic reaction.

The terms “plant-derived oil” mean a substance extracted from a plant or seed that exists in liquid form at room temperature. Suitable plant-derived oils include, without limitation, palm, olive, corn, canola, coconut, soybean, wheat germ, jojoba, sunflower, sesame, peanut, cottonseed, safflower, soybean, rapeseed, almond, beech nut, cashew, hazelnut, macadamia, mongongo nut, pecan, pine nut, pistachio, walnut, grapefruit seed, lemon, orange, bitter gourd, bottle gourd, buffalo gourd, butternut squash seed, egusi seed, pumpkin seed, watermelon seed, acai, black seed, blackcurrant seed, borange seed, evening primrose, flaxseed, eucalyptus, amaranth, apricot, apple seed, argan, avocado, babassu, coriander seed, grape seed, mustard, poppyseed, rice bran, castor, or mixtures thereof. Mixtures can be, by way of example and without limitation, a combination of olive oil and soybean oil, a combination of coconut oil and wheat germ oil, or a combination of jojoba oil, palm oil, and castor oil. Mixtures of suitable oils can be binary, ternary, quaternary, or higher mixtures.

The terms “resistant Candida strain” mean one or more species of Candida that is resistant to known clinical treatments to treat Candida infections such as, for example, topical applications of pharmacological effective antimicrobial active materials. Resistant Candida auris, for example, could refer to this Candida species that is resistant to known topical applications of pharmacological effective amounts of fluconazole.

The term “topical” refers to the application of the composition to any skin or mucosal surface. “Skin surface” refers to the protective outer covering of the body of a vertebrate, generally comprising a layer of epidermal cells and a layer of dermal cells. A “mucosal surface,” as used herein, refers to a tissue lining of an organ or body cavity that secretes mucous.

The terms “antibacterial”, “anti-fungal”, or “anti-protozoan” refer to inhibition or arrest of the growth of a bacterium, fungus, or protozoans, or a reduction in the severity of or likelihood of developing a bacterial, fungal, or protozoan disease, inducing death of the bacterium, fungus, or protozoans, or reduction or inhibition of the pathogenic effects of the respective bacterium, fungus, or protozoans. “Bactericidal” is used interchangeably with “antibacterial.”

The terms “treat”, “treatment”, and “treating” refer to an approach for obtaining beneficial or desired results, for example, clinical results. For the purposes of this disclosure, beneficial or desired results may include inhibiting or suppressing the growth of a microorganism or killing a microorganism; inhibiting one or more processes through which a microorganism infects a cell or patient; inhibiting or ameliorating the disease or condition caused by a microbial infection; or a combination thereof. The terms “treat”, “treatment”, or “treating” also refer to prophylaxis treatment. “Prophylaxis” refers to prevention of an infection or disease, or prevention of the development of symptoms of that infection or disease, a delay in the onset of an infection or disease or its symptoms, or a decrease in the severity of a subsequently developed infection or disease or its symptoms.

In one embodiment, the composition provided herein comprises the monoglyceride, GML. GML is a fatty acid ester of glycerol, derivative of lauric acid, with the chemical formula C₁₅H₃₀O₄. GML is also known in the art as glyceryl laurate or monolaurin. GML is found naturally in breast milk and some plants, and is used as a food and cosmetic additive. GML and other glycerides are listed in the Generally Recognized as Safe Substances database by the US Food and Drug Administration. GML and related compounds have been disclosed in U.S. patent Publication Nos. 2007/0276049, 2016/0175244, and 2017/0172968; and in U.S. patent Nos. 8,796,332, 9,603,824 and 9,724,295.

GML can be obtained or synthesized in multiple forms including both R and S optical isomers, as well as forms with lauric acid in the 1/3-position and in the 2-position. The gel-based formulation provided herein, in one embodiment, comprises the R isomer of GML. In another embodiment, the formulation comprises the S isomer of GML. In yet another embodiment, the formulation comprises a racemic mixture of isomers.

Similarly, the formulation may comprise GML with lauric acid ester at the 1/3 position, GML with lauric acid ester at the 2-position, or a combination thereof. R and S isomers of each form and racemic mixtures thereof, are amenable for use with the present invention.

The chemical structure of GML with lauric acid in the 1 or 3 positions is glycerol monolaurate (GML) 1/3-position.

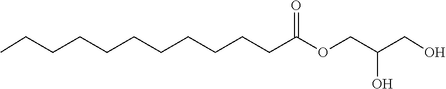 [SMILES: CCCCCCCCCCCC(=O)OCC(O)CO]

The chemical structure of GML with lauric acid ester in the 2-position is: Glycerol monolaurate (GML) 2-position.

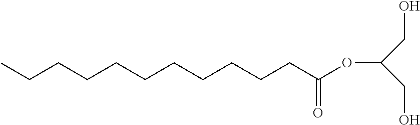 [SMILES: CCCCCCCCCCCC(=O)OC(CO)CO]

In another embodiment, the gel-based formulation comprises a GML derivative, for example a compound selected from one of Formulas A-F. Examples of such compounds include, by way of example and without limitation, glycerol monocaprylate, glycerol monocaprate, glycerol monomyristate, glycerol monopalmitate, and dodecyl glycerol.

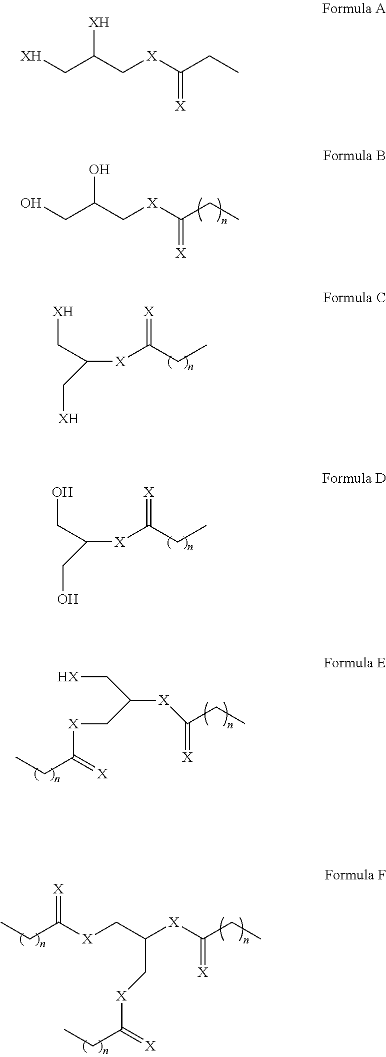

wherein each occurrence of X is independently —O— or —S—; and n is an integer from 5 to 20 (inclusive).

In another embodiment, the gel-based formulation comprises at least one derivative of GML, and the at least one derivative is a compound of either Formula E or Formula F. Examples of such compounds include, but are not limited to, glycerol dilaurate, glycerol dicaprylate, glycerol dimyristate, glycerol trilaurate, and glycerol tripalmitate.

In one embodiment, a compound of Formula A, B, C, or D is present in a formulation of this disclosure, and at least one —X— is —S—. In one embodiment, one occurrence of —X— is —S— and the remaining occurrences of —X— are —O—.

In one embodiment, a compound of Formula E or F is present in the formulation of This disclosure, each occurrence of n is 10, and at least one —X— is —O—.

The gel-based formulation provided herein, in one embodiment, comprises GML and/or a GML derivative. For example, in one embodiment, the gel-based formulation provided herein comprises GML and a compound of Formula F. In a further embodiment, each occurrence of n is 10 and at least one —X— is —O—.

In another embodiment, the gel-based formulation comprises GML or derivative thereof at a concentration of about 10 μg/mL to about 100 mg/mL. In a further embodiment, the gel-based formulation comprises GML or derivative thereof at a concentration of about 50 μg/mL to about 50 mg/mL. In a further embodiment, the gel-based formulation comprises GML or derivative thereof at a concentration of about 100 μg/mL to about 10 mg/mL. In yet a further embodiment, the gel-based formulation comprises GML or a derivative thereof at a concentration of about 500 μg/mL to about 5 mg/mL.

In one embodiment, the gel-based formulation comprises GML or derivative thereof at a concentration of about 10 μg/mL, about 50 μg/mL, about 100 μg/mL, about 500 μg/mL, about 1 mg/mL, about 5 mg/mL, about 10 mg/mL, about 50 mg/mL, or about 100 mg/mL.

Exemplary GML Concentrations

| | | |
|---|---|---|
| 0.001% | 10 | μg/mL |
| 0.01% | 100 | μg/mL |
| 0.1% | 1 | mg/mL |
| 1% | 10 | mg/mL |
| 2.5% | 25 | mg/mL |
| 5% | 50 | mg/mL |
| 7.5% | 75 | mg/mL |
| 10.0% | 100 | mg/mL |

The amount of GML or derivative thereof in the composition can be tailored accordingly to the extent of the urinary tract infection being treated as well as the characteristics of the patient being treated. The amount of GML in the composition may vary depending on, for example, the nature of the infection or illness; the site of administration; the patient's medical history, patient weight, age, sex, and surface area being treated; and whether the patient is receiving any other medications.

As provided above, in one aspect, the present disclosure is directed to a gel-based formulation comprising GML or a derivative thereof. In one embodiment, the gel-based formulation comprises at least one glycol. For example, in one embodiment, the gel-based formulation comprises propylene glycol, polyethylene glycol, or a combination thereof. In one embodiment, the polyethylene glycol has a molecular weight (MW) range from about 300 to about 10,000. In a further embodiment, the polyethylene glycol has a molecular weight of about 300 to about 1,000. In a still further embodiment, the polyethylene glycol has a molecular weight of about 400.

In one embodiment, polyethylene glycol is present in the gel-based formulation. In a further embodiment, the polyethylene glycol has a MW of about 400, about 500 or about 1,000. In one embodiment, the polyethylene glycol is present in the gel-based formulation at a concentration (w/w) of about 15% to about 50%, about 20% to about 40%, or about 25% to about 35%, for example, about 15%, about 20%, about 25%, about 30%, about 35%, about 40%, about 45%, or about 50%. In a further embodiment, both propylene glycol and polyethylene glycol are present in the gel-based formulation. In a further embodiment, propylene glycol is present at a concentration of about 70% to about 80% and polyethylene glycol is present at a concentration of about 20% to about 30%. In even a further embodiment, the polyethylene glycol is polyethylene glycol 400.

In a further embodiment, propylene glycol is present in the composition. In yet a further embodiment, propylene glycol is present in the composition at a concentration of about 60% to about 80%, for example, about 60%, about 65%, about 70%, about 71%, about 72%, about 73%, about 74%, about 75%, or about 80%.

In another embodiment, a gel-based formulation comprising GML or a derivative thereof is provided. In one embodiment, the gel-based formulation comprises at least one cellulose derivative. In a further embodiment, the composition comprises one cellulose derivative or two cellulose derivatives. In one embodiment, the cellulose derivative is hydroxypropyl cellulose. In another embodiment, the cellulose derivative is hydroxyethyl cellulose, carboxymethyl cellulose or hydroxymethyl cellulose. In yet another embodiment, the composition comprises a combination of hydroxyethyl cellulose and hydroxypropyl cellulose. In one embodiment, the cellulose derivative is present at a concentration of about 0.1% (w/w) to about 5.0% (w/w). In a further embodiment, multiple cellulose derivatives are present in the composition at the same concentration. In a further embodiment, two cellulose derivatives are present, and each is present at a concentration of about 1.25% (w/w).

Cellulose derivatives include, for example, hydroxyethyl cellulose, hydroxypropyl cellulose, methylcellulose, ethylcellulose, hydroxypropyl methyl cellulose, or cellulose acetate.

In one embodiment, the gel-based formulation provided herein comprises GML or a derivative thereof, at least one cellulose derivative, propylene glycol and polyethylene glycol.

In another embodiment, a gel-based formulation comprising GML or a derivative thereof is provided. In a further embodiment, the composition comprises at least one plant-derived oil, for example, at least one of the oils described above (e.g., palm oil, olive oil, or corn oil). In one embodiment, the plant-derived oil is present in the composition at a concentration of as much as about 100 w/w %.

In one embodiment, the gel-based formulation provided herein comprises a plant-derived oil and at least one cellulose derivative. For example, in one embodiment, the gel-based formulation comprises hydroxypropyl cellulose and a plant-derived oil, or hydroxyethyl cellulose and a plant-derived oil, or a combination of hydroxypropyl cellulose, hydroxyethyl cellulose, and a plant-derived oil. In one embodiment, the cellulose derivative and the plant-derived oil (e.g., palm oil, corn oil, or plant oil), are each present at the same concentration (w/w). In another embodiment, the gel-based formulation comprises petroleum jelly. In one embodiment, the composition comprises a plant-derived oil and two cellulose derivatives. In a further embodiment, the two cellulose derivatives are hydroxypropyl cellulose and hydroxyethyl cellulose, and the total concentration of cellulose derivatives in the composition is about 1.25% (w/w). Cellulose derivatives include, for example, hydroxyethyl cellulose, hydroxypropyl cellulose, methyl cellulose, ethyl cellulose, hydroxypropyl methyl cellulose, or cellulose acetate.

In some embodiments, the gel-based formulation provided herein comprises one or more accelerants. In a further embodiment, the accelerant is an organic acid, a chelator, or a combination thereof. In a further embodiment, the accelerant is a chelator. In even a further embodiment, the accelerant is EDTA.

The accelerant, in one embodiment, is EDTA. In a further embodiment, the GML composition provided herein comprises EDTA at a concentration of about 0.00005 M, about 0.0005 M, about 0.005 or about 0.05 M. In another embodiment, a chelator is present in the composition at a concentration of about 0.00005 M to about 0.05 M, about 0.0005 M to about 0.005 M, or about 0.005 to about 0.05 M.

In one embodiment, the gel-based formulation comprises both a plant-derived oil and an accelerant, for example palm oil and EDTA. In another embodiment, the accelerant is an organic acid and is present in the formulation with a plant-derived oil. In one embodiment, the gel-based formulation provided herein comprises an accelerant and a non-aqueous gel, for example a gel comprising a cellulose derivative. In another embodiment, the gel-based formulation comprises GML or a derivative thereof, a plant-derived oil, a non-aqueous gel (e.g., a gel comprising one or more cellulose derivatives) and an accelerant.

In one embodiment, the composition contains at least one pharmaceutically acceptable excipient. Pharmaceutically acceptable excipients are well known to those skilled in the art and may include buffers (e.g., phosphate buffer and citrate buffer), amino acids, alcohols, proteins such as serum albumin, parabens (e.g., methylparaben), or mannitol.

In one embodiment, the pH of the composition is from about 3.5 to about 7.0. In a further embodiment, the pH of the composition is from about 4.0 to about 6.0. In a still further embodiment, the pH of the composition is from about 4.0 to about 4.5.

In one embodiment, the composition provided herein comprises GML or a derivative thereof and a pharmaceutically acceptable topical carrier. In one embodiment, the pharmaceutically acceptable topical carrier is a mix of hydrocarbons such as, for example, paraffin wax or petroleum jelly. Petroleum jelly is any water-insoluble, hydrophobic, semi-solid mixture of hydrocarbons. The pharmaceutically acceptable topical carrier can be added to any of the formulations described herein.

In one embodiment the gel-based formulation comprises an additional active material. Additional active materials include, for example, antibacterial, anti-viral, anti-fungal, and anti-protozoan materials. Antibacterial materials include, without limitation, aminoglycosides, carbacephems, cephalosporins, glycopeptides, lincosamides, lipopetides, macrolides, monobactams, nitrofurans, penicillins, polypetides, quinolones, sulfuramides, or tetracyclines. Anti-fungal materials include, without limitation, those of the azole class, polyene class, or echinocandins class, nucleoside analogues, allylamines, griseofulvin, tolnaftate, or selenium compounds. Anti-viral materials include, for example and without limitation, acyclovir, ganciclovir, valganciclovir, abacavir, enofovir, lamivudine, emtricitabine, zidovudine, tenofovir, efavirenz, raltegravir, enfuvirdide, maraviroc, ribavirin, amantadine, rimantadine, interferon, oseltamivir, or zanamivir.

In another embodiment, the composition is a solid, semi-solid, foam, wax, cream, or lotion.

The GML gel-based formulations described herein may be less irritating than currently approved antimicrobial compositions, therefore resulting in a more favorable patient compliance rate, as compared to other antimicrobial compositions presently used in the art.

In one embodiment, the method comprises administering to the patient a gel-based formulation comprising GML or a derivative thereof, as described herein. In one embodiment, the method comprises topically administering to the patient an effective amount of a composition comprising GML or a derivative thereof, a plant-derived oil, and a pharmaceutically acceptable topical carrier. In another embodiment, the method comprises topically administering an effective amount of a composition comprising GML, a non-aqueous gel, and a pharmaceutically acceptable topical carrier. For example, the composition may be given twice per day for 3-4 days, or 6-7 days. Alternatively, the composition may be given once per day for 7-10 days or 12-14 days.

In one embodiment, the method of treating a microbial infection comprises applying an effective amount of one or more of the GML compositions described herein to at least one skin or mucosal surface of a patient.

In some embodiments, the gel-based formulation is applied to or impregnated in a wipe, sponge, swab, or other material, and then applied to the skin or mucosal surface of the patient using the respective material. As used herein, the term “swab” refers to a material suitable for applying a liquid, gel, wax, cream, or lotion to a skin or mucosal surface, or the act of applying a liquid, gel, wax, cream, or lotion to the skin or mucosal surface, or the act of collecting a liquid, gel, wax, cream, lotion, or fluid from the skin or mucosal surface. In some embodiments, the material is attached to a holder, for example a stick, wire, rod, or applicator. In further embodiments, the material attached to a holder is attached at one or both ends thereof. In some embodiments, the wipe, sponge, swab, or other material is pre-loaded or packaged together with the composition.

In other embodiments, the gel-based formulation is applied to or impregnated in an implanted, or other indwelling, device and the coated device is then placed in a patient using known processes and procedures.

GML compositions inhibit microbial infection through one or more of several mechanisms that include, but are not limited to, direct microbial toxicity; inhibiting entry of the infectious microorganism into the vertebrate cell; inhibiting growth of the microorganism; inhibiting production or activity of virulence factors such as toxins; stabilizing the vertebrate cells; or inhibiting induction of inflammatory or immunostimulatory mediators that otherwise enhance the infectious process.

In one embodiment, direct GML-mediated interruption of bacterial membranes includes interference with the localization of signaling proteins within the membrane, or interference with ligand binding to signaling proteins. In one embodiment, GML has an indirect effect on a two-component signal transduction system and the effect is selected from modifications to membrane structure that interfere with the ability of transmembrane proteins to perform signaling functions; dissipation of the bacterial plasma membrane potential; and alterations of pH gradients across the membranes.

Similar to GML's putative effects on bacterial plasma membranes, GML has been shown to inactivate certain viruses by disrupting viral lipid envelopes.

Methods of identifying and diagnosing a bacterial, viral, fungal, or protozoan infection are generally known by those skilled in the art. To assess whether the formulations disclosed herein are useful to treat an infection, methods known to those of ordinary skill in the art may be employed.

In one embodiment, a method is provided to remove or kill a biofilm comprising one or more microorganisms. In one embodiment, the method comprises administering the gel-based formulation by applying it directly to the biofilm. In some embodiments, the methods of this disclosure comprise administering a second active material, along with GML or a derivative of GML. The additional active material may be present in the compositions described herein, or may be administered separately. In one embodiment, the one or more additional active materials prior to, or after, the topical GML composition is administered. For example, the two active materials may be topically administered serially, or administered serially by different routes of administration.

In one embodiment, the additional active material (s) is administered before, during, or after administration of the composition of this disclosure. In another embodiment, the additional active material(s) is administered by the same route as the composition or by a different route. For example, the additional active material(s), in one embodiment, is administered by one of the following routes of administration: topical, intranasal, intradermal, intravenous, intramuscular, oral and subcutaneous. The dose of additional active materials depends on, for example, the nature of the infection or illness; the site of administration; patient weight, age, sex, and surface area; concomitant medications; and medical judgment.

### EXAMPLES

The present disclosure is further illustrated by reference to the following Examples. However, it should be noted that these Examples, like the embodiments described above, are illustrative and are not to be construed as restricting the scope of the claims in any way.

In the Examples, the Candida species used were all clinical isolates.

### Example 1

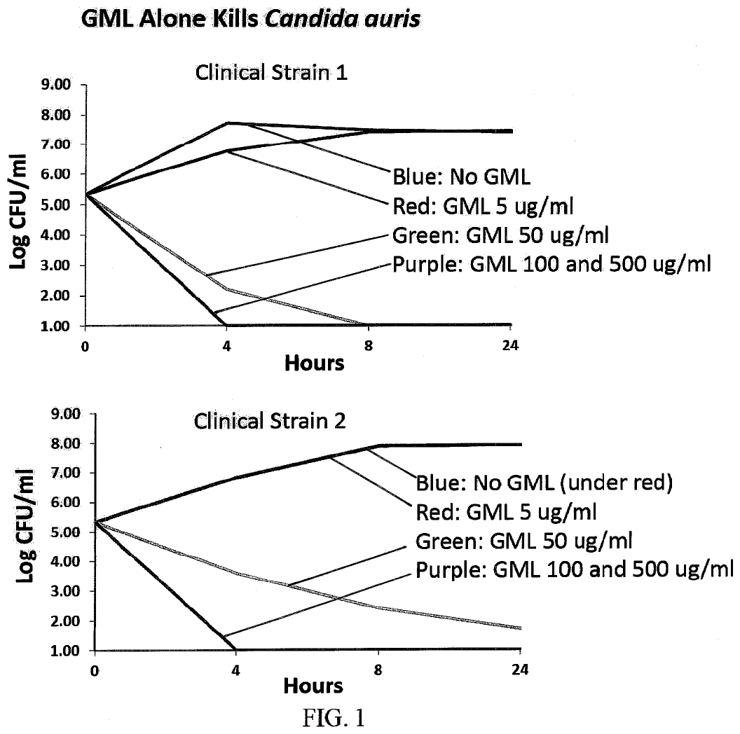

In a first study performed was to test the ability of GML alone to kill two recent clinical isolates of C. auris (FIG. 1). The two strains individually were added to Todd Hewitt growth medium (25 ml in 125 ml Erlenmeyer flasks. The starting inoculum as determined by plate counts on chocolate agar was approximately 105 colony-forming units (CFU)/ml. The cultures were incubated at 37° C. for periods up to 24 hours, with sampling at 0, 4, 8, and 24 hours. CFUs/ml were determined on chocolate agar at each time point in triplicate. The data obtained are reported in log CFUs/ml. Although not presented, the standard deviations were determined and were all ≤0.02. The data in FIG. 1 show that C. auris is killed by GML alone in growth medium by 8 hours' incubation (significant killing is defined as ≥3 logs killing) at GML concentrations of ≥50 μg/ml. The lower limit of detection of Candida is 10 CFUs/ml for these studies.

### Example 2

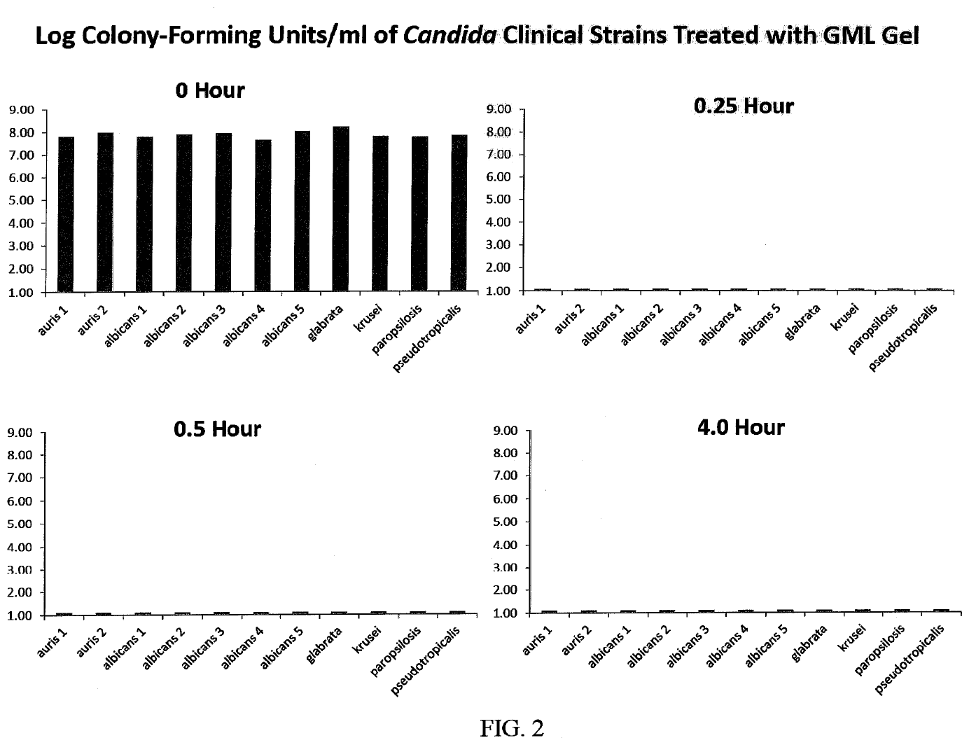

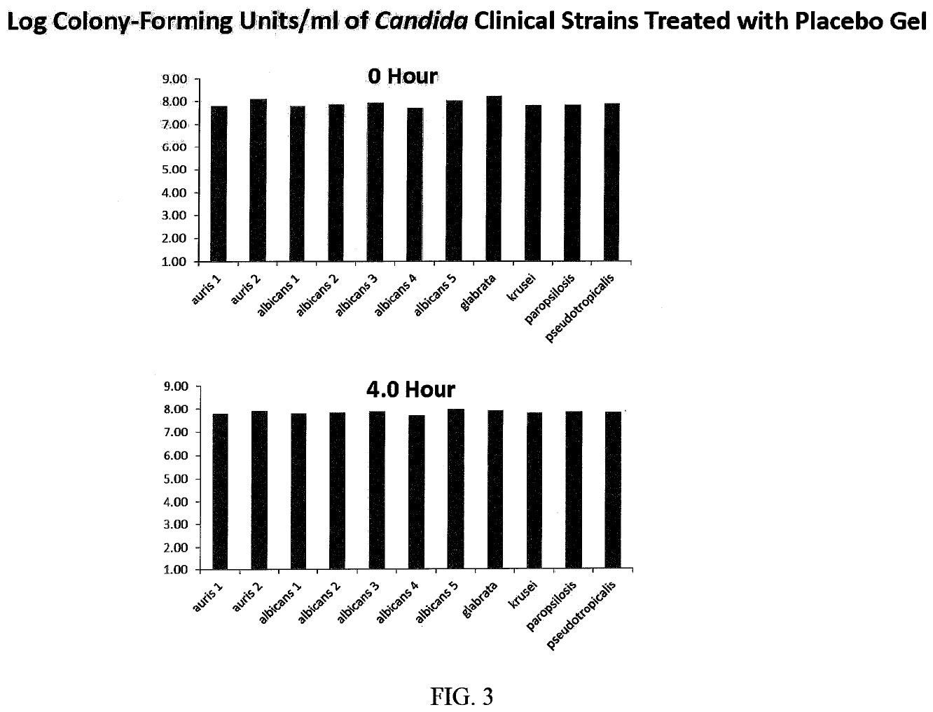

In a second study, all pathogenic species of Candida were evaluated for killing by GML formulated as a single antimicrobial in an non-aqueous gel; called 5% GML Gel. This is the first time such a study has been done. Multiple strains of C. albicans, two strains of C. auris, and one strain each of the remaining pathogens were tested by adding the microbes at approximately 108 CFUs/ml in a 0.1 ml volume to 5 ml of 5% GML Gel. The tubes containing the mixtures were inverted 5 times and then incubated at 37° C., which is body temperature and decreases the viscosity of the GML Gel. Data were compared to the universal HIV universal placebo which contains 2.7% hydroxyethyl cellulose and normal saline with pH adjusted to 4.5. The data for 5 GML Gel are shown in FIG. 2 and data for placebo gel are shown in FIG. 3. All CFUs/ml were determined in triplicate on chocolate agar. By 15 minutes post exposure to 5% GML Gel, all Candida species were killed to below detection levels (the lower limit of detection is 10 CFUs/ml). The same data were obtained at 30 minutes and 4 hours incubation. In contrast, the placebo gel was not antimicrobial when tested at 4 hours.

### Example 3

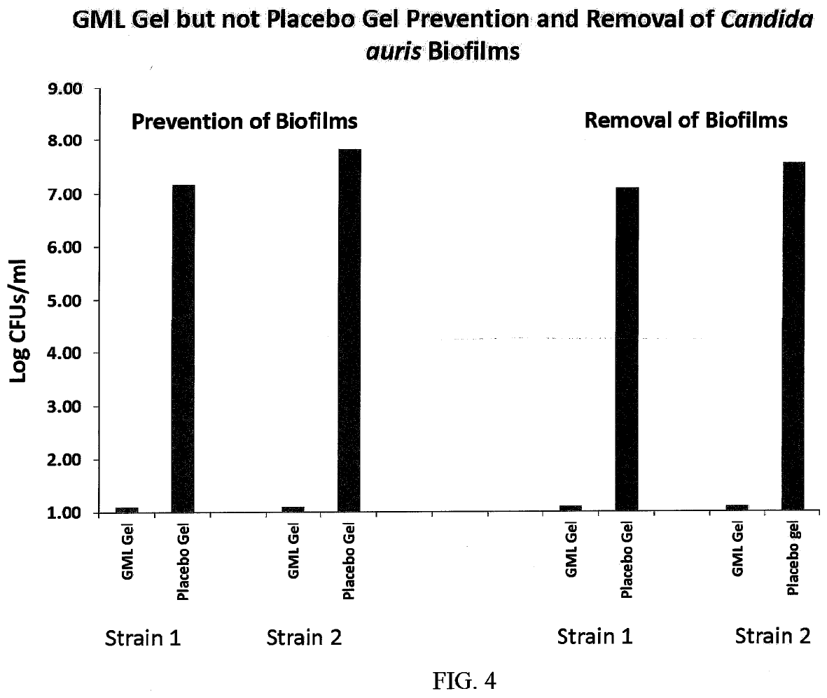

In a third study, two C. auris strains (the two strains used in Example 2) were added to 96 well microtiter plates (0.2 ml of 2.5-4.9×105/ml) in triplicate Immediately after addition of strains, 3 wells for each were treated with 0.1 ml of 5% GML Gel or 0.1 ml of placebo gel. The wells were then incubated at 30° C. for 24 hours. In another set of 3 wells each, the C. auris strains were cultured for 48 hours to allow biofilms to form. Then, 5% GML Gel or placebo gel (0.1 ml) was added to each well. The wells were incubated for an additional 8 hours at 37° C. Then, plate counts were performed to assess CFUs/ml. As shown in FIG. 4, no detectable Candida grew from wells treated immediately (to prevent biofilm formation) or after biofilms were allowed to form for 48 hours. This indicates the 5% GML Gel was effective in killing the organisms to prevent and remove biofilms. The lower limit of detection in this assay was 10 CFUs/ml. In contrast, the placebo gel did not inhibit growth of the C. auris strains and did not remove and kill strains already in biofilms.

Modifications and variation of the above-described embodiments of this disclosure are possible, as appreciated by those skilled in the art in light of the above teachings. It is therefore understood that, within the scope of the claims and equivalents of this disclosure, the described invention may be practiced otherwise than as specifically described.

1. A method of treating an patient with an infection associate with an antimicrobial resistant Candida strain comprising the step of administering a composition comprising a pharmaceutically effective amount of glycerol monolaurate or a derivative to the infected patient for a sufficient amount of time to kill the resistant Candida strain.

2. A method of treating a biofilm of an antimicrobial resistant Candida strain in an infected patient comprising the step of administering a pharmaceutically effective amount of glycerol monolaurate or a derivative to the biofilm in the infected patient for a sufficient amount of time to clinically remedy the resistant Candida strain biofilm.

3. The method of claim 1, wherein administering glycerol monolaurate or a derivative is topical, oral or by injection.

4. The method of any of claim 1, wherein the Candida strain is resistant to antifungal agents.

5. The method of claim 1, wherein the Candida strain is resistant to fluconazole.

6. The method of claim 1, wherein the glycerol monolaurate or a derivative is in a solution in an amount of ≥50 μg/ml.

7. The method of claim 1, wherein glycerol monolaurate or a derivative is in a non-aqueous gel in an amount of about 5 wt %

8. The method of claim 1, wherein the glycerol monolaurate or a derivative is an aqueous ointment, cream, lotion.

9. The method of claim 1, wherein ≥3 logs of resistant Candida species are killed in less than 15 minutes after administering glycerol monolaurate or a derivative.

10. The method of claim 1, wherein the resistant Candida species is reduced from ≥8 logs to less than 10 CFU/ml

11. Th method of claim 1, wherein the resistant Candida species is Candida auris.

12. The method of claim 1, wherein the infection is a vaginal infection, thrush, or a blood infection.

13. The method of claim 1, wherein the glycerol monolaurin is a gel-based formulation comprising comprises about 10-100 mg/mL glycerol monolaurate, about 0.0001-0.05 M of ethylenediaminetetraacetic acid, and a topical solution comprising a) about 73.55 w/w % propylene glycol; b) about 25 w/w % polyethylene glycol 400; c) about 1.25 w/w % hydroxyethyl cellulose or hydroxypropyl cellulose; and d) about 1-25 w/w % saline and/or water.
